(19) United States
(12) Patent Application Publication
(10) Pub. No.: US 2017/0071846 A1
(43) Pub. Date: Mar. 16, 2017

(54) EXFOLIANT WITH BIOSURFACTANTS

(71) Applicant: Henkel AG & Co. KGaA, Duesseldorf (DE)

(72) Inventors: Heike Schelges, Willich (DE); Maria Tretyakova, Dortmund (DE)

(73) Assignee: Henkel AG & Co. KGaA, Duesseldorf (DE)

(21) Appl. No.: 15/254,781

(22) Filed: Sep. 1, 2016

(30) Foreign Application Priority Data
Sep. 14, 2015 (DE) 10 2015 217 504.9

(51) Int. Cl. A61K 8/81; A61K 8/25; A61K 8/73; A61K 8/92; A61K 8/41; A61K 8/60; A61Q 19/10; A61K 8/29

(52) U.S. Cl. CPC A61K 8/8152; A61Q 19/10; A61K 8/25; A61K 8/29; A61K 8/922; A61K 8/41; A61K 8/602; A61K 8/73; A61K 8/737; A61K 2800/48; A61K 2800/5922; A61K 2800/596; A61K 2800/594; A61K 2800/28; A61K 2800/412

(57) ABSTRACT

Exfoliants, i.e. cosmetic cleansers, include an abrasive component. The exfoliants according to the invention include biosurfactants as surfactants and also include a natural thickening agent. The abrasive component, the biosurfactants, and the thickening agent of the exfoliants according to the invention are based on natural, renewable and biologically degradable raw substances.

### FIELD OF THE INVENTION

The present invention generally relates to exfoliants, i.e. cosmetic cleansers that include an abrasive component.

### BACKGROUND OF THE INVENTION

The cleansing and care of healthy, smooth skin having no, or only minor, impurities usually provides no problems. By contrast, the cleansing of blemished or oily skin or of combination skin is problematic, since many active substances used to combat blemished or oily skin act in a very heavily degreasing manner and can provoke an even quicker production of sebum. The problem of blemished skin can be aggravated even further as a result.

What are known as exfoliants constitute a variant of cosmetic cleansers for use on blemished skin that is particularly preferred by many consumers. With exfoliants, the microcirculation of the skin can be increased, which gives the skin a radiant appearance.

Cosmetic exfoliant products known in the prior art generally include abrasively acting particles, such as polyethylene powder, walnut husk powder, apricot or almond kernel powder, wherein the function of the abrasively acting components is essentially the removal of dead flakes of skin, but also of excess sebum and of fatty substances on the skin by means of friction. DE 102013209894 A1 for example discloses exfoliants in which a combination of polylactic acid particles and other, known particles having an abrasive effect is used as abrasive component. In WO 2011/149689, foaming body exfoliants are described which include biologically degradable polylactic acid particles as abrasive component. On account of their excellent cleansing and foaming capability, these and further conventional cosmetic cleansers, including exfoliants, include predominantly anionic surfactants, possibly in mixture with small amounts of co-surfactants.

A large number of commercially available anionic surfactants soften the skin during the cleansing process and remove lipids from the outer skin layers. The skin can thus become dry, rough and sometimes cracked, which is undesirable in particular in the case of exfoliants. On the other hand, anionic surfactants often cannot be fully replaced by milder (for example non-ionic) surfactants, because the cleansing and foaming effect of the product is reduced as a result.

Furthermore, sustainable bodycare products are being increasingly demanded by consumers, i.e. in particular products that are obtained from renewable raw substances that are as natural as possible and that are biologically degradable.

Biosurfactants are surface-active substances of microbial origin that can be produced on the basis of vegetable oil substrates or sugar substrates. These substrates can consist in part of agricultural waste, such as rice husks or wastewaters from the sugar industry, such that, in this case as well, no starting materials for food production are lost. Biosurfactants thus meet the requirements of sustainability, since they are produced from renewable raw materials. They are used both in domestic cleaning agents, detergents, and dishwashing detergents (for example U.S. Pat. No. 5,520,839, DE 19600743 A1) and also in various cosmetic cleansers (for example WO 2014/095367 A1, WO 2013/098066 A2). However, biosurfactants alone generally have a low foaming capability.

EP 2037942 B1 (WO 2007/143006 A1) discloses the use of special biosurfactants in cosmetic cleansers, which can include abrasive components. There is no disclosure of a specific exfoliant composition.

There is a need for exfoliants that are gentle on the skin, that are produced largely or exclusively from natural, renewable and biologically degradable products, and that nevertheless meet demands on foaming behaviour and cleansing and care behaviour of currently obtainable exfoliants.

Furthermore, other desirable features and characteristics of the present invention will become apparent from the subsequent detailed description of the invention and the appended claims, taken in conjunction with this background of the invention.

### BRIEF SUMMARY OF THE INVENTION

In the exfoliants according to the invention, biosurfactants are included as surfactants. The abrasive component, the biosurfactants, and further constituents of the exfoliants according to the invention are based on natural, renewable raw materials.

A cosmetic cleanser includes the following constituents, in each case in wt. % in relation to the total weight: 0.1 to 20 wt. % of at least one water-insoluble abrasive component of natural origin, 1 to 50 wt. % of at least one biosurfactant, and 0.2 to 3 wt. % of at least one thickening agent of natural origin.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has surprisingly been found that exfoliants can be provided which include predominantly or exclusively biosurfactants as surfactants, natural abrasive components, and also a natural thickening agent.

The present invention relates to a cosmetic cleanser including the following constituents, in each case in wt. % in relation to the total weight:

(a) 0.1 to 20 wt. % of at least one water-insoluble abrasive component of natural origin,
(b) 1 to 50 wt. % of at least one biosurfactant, and
(c) 0.2 to 3 wt. % of at least one thickening agent of natural origin.

The cosmetic product according to the invention can also be referred to hereinafter as an “exfoliant” and includes, as necessary constituent, at least one abrasive component. An abrasive component is understood to mean a component that is suitable, with use of the cosmetic product, for removing fatty substances and dirt on the skin by means of friction or abrasion.

In accordance with the invention, an abrasive component of natural origin is used as abrasive component. Here, in accordance with the invention, the term “of natural origin” is understood to mean that the abrasive component does not have to be synthetically produced, but that natural materials are used, which possibly are comminuted beforehand. Organic and inorganic materials are considered for this purpose. Examples of organic abrasive components of natural origin are (crystalline) cellulose, jojoba wax (jojoba wax beads), and crushed and/or ground plant parts, such as crushed and/or ground peach kernels, apricot kernels, walnut husks, orange kernels, bitter orange kernels, grape kernels (grape kernel exfoliator), lychee kernels (lychee exfoliator), almond kernels, cherry kernels, bamboo powder, cranberry seeds (cranberry exfoliator) and/or lotus seeds (lotus exfoliator), possibly dried fruit flesh of almonds, coconuts, jojoba fruits, macadamia nuts and other nuts, almond meal, wheat bran, oatmeal, sawdust (wood powder) and nutshell powder, in particular walnut shell powder or corncob powder, ginger powder, green tea powder, hibiscus seed powder, star anise powder, loofah powder, and crushed or ground algae obtainable under names such as lithothamnium, Fucus micronised, Spirulina micronised, and also carbon. Jojoba wax, cellulose, comminuted plant parts, in particular comminuted fruit stones and/or algae, and combinations thereof are particularly preferred.

By way of example, clay earths, talcum powder, zeolites, rhyolite, sand, sea sand, pumice stone powder, chalk, shell limestone and/or marble powder are suitable inorganic abrasive components. Sand, silicon dioxide, and titanium dioxide are particularly preferred. See sand is especially preferred.

The absolute particle sizes of the organic or inorganic abrasive components can lie preferably in the range from 40 to 500 μm. The use of smaller particle sizes leads to an overall impaired effect of the cosmetic cleansers according to the invention, since abrasive components of a particularly small particle size—in particular when used on skin that has large pores—sometimes led to blocking of the skin pores. A blocking of the skin pores can result in inflammatory reactions, which is undesirable.

The use of larger particle sizes likewise led to an impaired effect of the cleansers according to the invention, since the abrasive effect of the particles was too severe and can lead to inflammation of the skin and minor injuries to the skin.

Particle sizes in the range from 40 to 400 μm, preferably from 50 to 300 μm, particularly preferably from 60 to 250 μm, and especially preferably from 70 to 200 μm, are therefore particularly preferred.

The insolubility in water of the abrasive components is then important within the scope of the present invention when the cleansers according to the invention have an aqueous or aqueous-alcoholic carrier, in which the abrasive components should not be dissolved during storage. In a preferred embodiment the cleansers according to the invention have an aqueous carrier, and therefore water-insoluble components are required as abrasive components.

Polylactic acid particles can be included as further suitable and preferred abrasive component of natural origin. Polylactic acid, also referred to as polylactide or PLA, is one name for biologically degradable polymers (polyesters) that are accessible above all by the ionic polymerization of lactide, an annular combination of two lactic acid molecules.

At temperatures between 140 and 180° C. and also under the effect of catalytic tin compounds (for example tin oxide), a ring-opening polymerization occurs. Plastics having a high molecular mass and strength are thus produced. Lactide itself can be produced by fermentation of molasses or by fermentation of glucose with the aid of various bacteria.

In addition, high-molecular and pure polylactides can be produced directly from lactic acid with the aid of what is known as polycondensation. In industrial production, however, the disposal of the solvent is problematic.

The lactic acid (2-hydroxypropanoic acid) has an asymmetrical C atom, and therefore the polylactic acid also has optically active centres in L(+) and D(−) configuration. The ratio of L to D monomer units here determines the degree of crystallinity, the melting point, and the biological degradability of the polymers.

Polylactic acids that are suitable in accordance with the invention are L-polylactic acid, D-polylactic acid, and L/D polylactic acid, and mixtures thereof. On account of its very good biological degradability, L-polylactic acid is particularly preferred. In a preferred embodiment of the present invention, the proportion by weight of L-lactic acid monomer units in the polylactic acid is greater than 50 wt. %, preferably greater than 80 wt. %, and in particular greater than 90 wt. %.

The molar mass of the polylactic acid suitable in accordance with the invention is preferably 1,000 to 1,000,000, preferentially 10,000 to 300,000, more preferably 50,000 to 250,000, and in particular 100,000 to 180,000 Dalton.

Polylactide acid particles that are suitable in accordance with the invention can be present both in spherical form and in the form of irregular particles. It is assumed that irregular forms intensify the abrasive effect of the polylactic acid particles. Further information with regard to the form and further properties of suitable polylactic acid particles is disclosed in detail in DE 102013209894 A1.

The polylactic acid particles that are used in accordance with the invention preferably have sizes that are defined by their surface-equivalent diameter (ISO 9276-6:2008(E) section 7), also referred to as the “Equivalent Circle Diameter ECD” (ASTM F 1877-05 Section 1 1 .3.2). The mean ECD of a particle totality is calculated as the average ECD of each individual particle of a particle totality of at least 10,000 particles, preferably of more than 50,000 particles, in particular of more than 100,000 particles, once particles with a surface-equivalent diameter (ECD) below 10 μm have been excluded from the measurement.

In a preferred embodiment of the present invention the polylactic acid particles have mean ECD values from 10 to 1,000 μm, preferably from 50 to 500 μm, more preferably from 100 to 350 μm, and in particular from 150 to 250 μm.

Irrespective of the mean particle size, cosmetic cleansers according to the invention in which the polylactic acid particles have absolute particle sizes from 1 to 1,000 μm, more preferably from 1 to 850 μm, particularly preferably from 1 to 750 μm, especially preferably from 1 to 500 μm, and in particular preferably from 1 to 300 μm, are preferred.

Polylactic acid particles that can be used with preference in the cleansers according to the invention are commercially obtainable (for example from the company Micro Powders, Inc. under the trade name Ecoscrub®). The commercial products Ecoscrub® 20PC, Ecoscrub® 50PC, Ecoscrub® 100PC, Ecoblue®5025 and Ecogreen® 5025 are particularly preferred. Ecoscrub® 20PC and Ecoscrub® 50PC are preferred in particular.

In the present invention, a combination of polylactic acid particles with one or more of the inorganic or organic abrasive components of natural origin, in particular a combination of polylactic acid particles with see sand or jojoba wax particles, is particularly preferred as abrasive component.

The exfoliant according to the invention includes the abrasive component(s) in an amount from 0.1 to 20 wt. %, preferably 1 to 15 wt. %, more preferably 2 to 15 or 5 to 12 wt. %, in relation to the total weight of the exfoliant. If a plurality of abrasive components are included, the percentages relate to the sum of all included abrasive components. If polylactic acid particles are included, these are preferably included in combination with another abrasive component, the polylactic acid particles being included preferably in an amount from 0.1 to 7 wt. %, more preferably 1 to 5 wt. %, and the other abrasive component being present preferably in an amount from 3 to 15 wt. %, more preferably 5 to 10 wt. %.

The cosmetic cleanser according to the invention includes one or more biosurfactants as further essential constituent.

Biosurfactants are understood to mean substances that are formed by microorganisms and that are often also removed from the cell. Just like conventional surfactants, biosurfactants are surface-active substances, which reduce the surface tension of liquids and thus promote the mixing of aqueous (hydrophilic) and water-repelling (hydrophobic) phases. Biosurfactants can be produced under mild production conditions that require a low energy consumption. They are generally easily biologically degradable and they are very highly compatible with the environment. In addition, they are not toxic and there are also no toxic by-products created as a result of their production. Raw substances used for their microbial production include carbohydrates, in particular sugars, such as glucose, and/or lipophilic carbon sources, such as fats, oils, partial glycerides, fatty acids, fatty alcohols, long-chain saturated or unsaturated hydrocarbons. The biosurfactants are, in accordance with the invention, preferably biosurfactants produced by fermentation.

The biosurfactants include glycolipids, lipopeptides, lipoproteins, fatty acids, phospholipids, neutral lipids, and polymeric surfactants (for example emulsan), which can all also be used in the present invention.

Glycolipids that can be used in the present invention are compounds in which one or more monosaccharide units are glycosidically bonded to a lipid component. Examples of glycolipids as biosurfactants that can be used in accordance with the invention are rhamnolipids, sophorolipids, mannosylerythritol lipids and trehalose lipids. Among these, rhamnolipids, sophorolipids, mannosylerythritol lipids and combinations thereof are preferred.

Rhamnolipids are obtained from bacteria of the Pseudomonas genus, in particular from Pseudomonas aeruginosa, preferably with growth on hydrophobic substrates, such as n-alkanes or vegetable oils. Further glycolipids, such as glucose lipids, cellobiose lipids or trehalose lipids, are produced from other microorganisms on different substrates. In accordance with the invention, mannosylerythritol lipids are also preferred glycolipid biosurfactants; these are produced from bacteria of the Pseudozyma sp., Candida Antarctica and Ustilago sp. genus.

Rhamnolipids according to the invention have the following general formula:

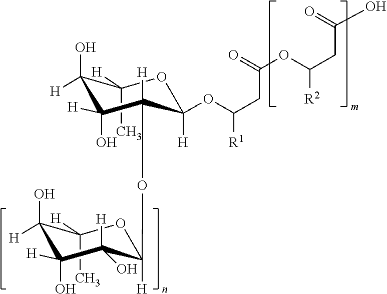

where m is 2, 1 or 0,
n is 1 or 0,
R¹ and R², independently of one another, are the same or different organic groups having 2 to 24, preferably 5 to 13 carbon atoms, in particular a substituted or unsubstituted, branched or unbranched alkyl group, which can also be unsaturated, the alkyl group preferably being a linear saturated alkyl group having 8 to 12 carbon atoms, more preferably a nonyl or decyl group or a mixture thereof.
Salts of these compounds are also included in accordance with the invention.

The term “di-rhamnolipid” in the present invention is understood to mean compounds of the above formula or salts thereof in which n is 1.

Accordingly, “mono-rhamnolipid” in the present invention is understood to mean compounds of the general formula or salts thereof in which n is 0.

Mixtures of mono- and di-rhamnolipids can be used with preference in accordance with the invention. Here, the ratio of mono-rhamnolipid to di-rhamnolipid is preferably approximately 2:1 to 4:1, more preferably 2.5:1 to 3:1. Mixtures of mono- and di-rhamnolipids in which, in the above formula, R¹ and R² independently of one another are a linear nonyl or a decyl group are particularly preferred. The latter case thus concerns rhamnolipids derived from 3-hydroxydodecanoic acid and/or 3-hydroxyundecanoic acid. Mixtures of this type are commercially obtainable for example under the name rhamnolipid R90, R95 or R98 from the company Algae Technologies, USA, the number specifying the degree of purity in each case. Rhamnolipid R90 can be used particularly preferably in accordance with the invention.

Sophorolipids are produced fermentatively with use of yeasts, such as Candida bombicola (also known as Torulopsis bombicola), Yarrowia lipolytica, Candida apicola (Torulopsis apicola), and Candida bogoriensis, by leaving these to grow on sugars, hydrocarbons, vegetable oils or mixtures thereof.

Sophorolipids have the formulas (1) (lacton form) and (2) (free acid) specified below, the two forms usually being present in a mixture.

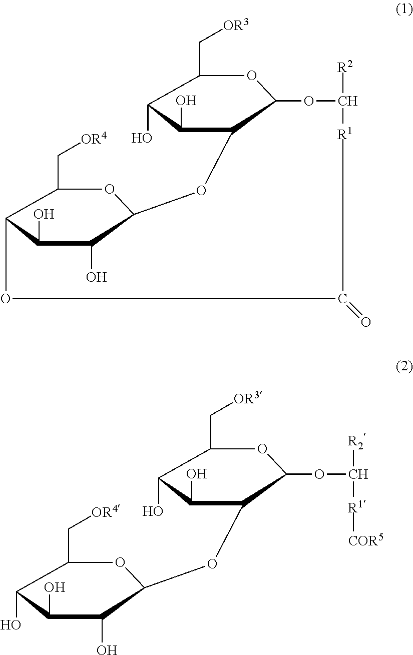

where R¹ and R^{1′}, independently of one another, are saturated hydrocarbon chains or mono- or polyunsaturated, in particular monounsaturated hydrocarbon chains having 8 to 20, in particular 12 to 18 carbon atoms, more preferably 14 to 18 carbon atoms, which can be linear or branched and can include one or more hydroxyl groups,
R² and R^{2′}, independently of one another, are a hydrogen atom or a saturated alkyl group or a mono- or polyunsaturated, in particular monounsaturated alkyl group having 1 to 9 carbon atoms, more preferably 1 to 4 carbon atoms, which can be linear or branched and can include one or more hydroxyl groups, and
R³, R^{3′}, R⁴ and R^{4′}, independently of one another, are a hydrogen atom or an acetyl group.

Sophorolipids in which R¹ and R^{1′} are a monounsaturated linear hydrocarbon chain having 15 carbon atoms are preferred. It is also preferred if R² and R^{2′} are a methyl group or a hydrogen atom, even more preferably are each a methyl group.

Sophorolipids in which the acid form and the lacton form are present in a mixture, where preferably approximately 20 to approximately 60 wt. % of the sophorolipid is present as acid form and the rest of the sophorolipid is present in the lacton form, are preferred in accordance with the invention.

In particular, preferred sophorolipids are those in which compounds of the above formulas (1) and (2) are present in a mixture, where R¹ and R^{1′} are monounsaturated linear hydrocarbon chains having 14 to 18 carbon atoms, even more preferably 15 carbon atoms, R³ and R⁴ are an acetyl group, R^{3′} and R^{4′} are a hydrogen atom, and R² and R^{2′} are a methyl group, and where approximately 20 to 60 wt. % of the sophorolipids are present in the acid form.

Sophorolipids of this type are commercially obtainable, for example under the name Sopholiance S from the company Soliance. More specifically, the sophorolipid obtainable under the trade name Sopholiance S from the company Soliance is an approximately 60 wt. % sophorolipid solution and is obtained for example by fermentation of Candida bombicola on rapeseed oil methyl ester and glucose (INCI: Candida bombicola/glucose/methyl rapeseed ferment (and) Water). Sopholiance S is a sophorolipid that is preferred in accordance with the invention.

In the case of Soliance S, the free acid form is present in an amount of approximately 20 wt. %, in a mixture with the lacton form.

Mannosylerythritol lipids are glycolipids of the following general formula:

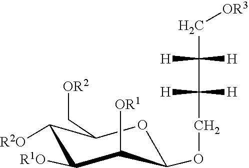

in which the R¹, independently of one another, are fatty acid acyl groups having 4 to 24 carbon atoms, preferably 8 to 12 carbon atoms, the R², independently of one another, are a hydrogen atom or an acetyl group, and R³ is a hydrogen atom or a fatty acid acyl group having 2 to 24 carbon atoms. A mannosylerythritol lipid that is suitable in accordance with the invention is commercially obtainable under the name Ceramela-B (Toyobo) (INCI: Pseudozyma Tsukubaensis/Olive Oil/Glycerin/Soy Protein Ferment).

The biosurfactants also include the substance group of lipids and lipid derivatives, which in particular include lipopeptides. Lipopeptides are generally synthesised non-ribosomally by the respective microorganisms, for example by gram-positive bacteria, in particular of the genus Pseudomonas, and by myxobacteria, and also by filamentous fungi. The peptide chains normally consist of two to forty amino acids and can be linear, cyclic or branched. In contrast to ribosomally synthesised peptide chains, they often do not only include proteinogenic L-amino acids as monomeric building blocks, but also D-amino acids and carboxylic acids and/or alpha-hydroxyl carboxylic acids of all types. The amino acids are usually L-α od D-α amino acids, however β, γ or δ amino acids can also be provided, which likewise can be present in D- or also in L-configuration. The peptide chains can also include further chemical modifications, in particular they can be glycosylated, hydrolysed, N-methylated or N-formylated. Frequently occurring structural elements are also thiazoline and/or oxazoline rings in various oxidations stages. A known lipopeptide biosurfactant is surfactin, which has the following structure and is generally used as an alkali or ammonium salt.

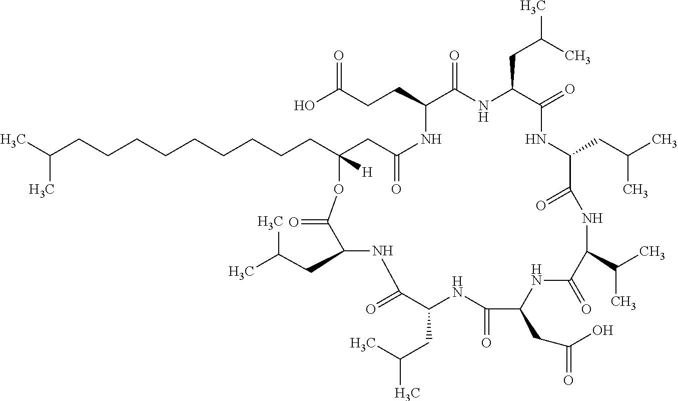 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

A surfactin that is suitable in accordance with the invention is commercially obtainable from the company Kaneka.

The lipopeptides that can be used with preference in accordance with the invention as biosurfactants also include what are known as Fatty Acyl Glutamates. These have the following formula:

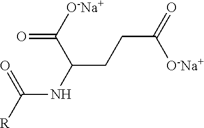

where R is a straight or branched alkyl chain having 5 to 21 carbon atoms, preferably 7 to 17 carbon atoms, more preferably 12 to 16 or 13 to 15 carbon atoms. Fatty Acyl Glutamates as biosurfactants are usually present in a mixture, in which R has different chain lengths. The group R can also be hydroxylated, preferably monohydroxylated, a hydroxylation at the β-position being preferred in this case. Fatty Acyl Glutamates as biosurfactants are obtainable for example from the company Modular Genetics, Inc., USA.

The lipopeptides that can be used with preference in accordance with the invention as biosurfactants also include what are known as Fatty Acyl Glycinates. These have the following general formula:

RC(O)NHCH₂CO₂X,

where
R is a straight or branched alkyl chain having 5 to 21 carbon atoms, preferably 7 to 17 carbon atoms, more preferably 12 to 16 or 13 to 15 carbon atoms, and
X is a cation, preferably an alkali metal or ammonium cation, more preferably a sodium or ammonium cation, or is —H.

Fatty Acyl Glycinates as biosurfactants can also be present in a mixture in which R can have different chain lengths.

Fatty Acyl Glycinates as biosurfactants are obtainable for example from the company Modular Genetics, Inc., USA.

In accordance with the invention, cosmetic cleansers that include the following biosurfactants are preferred: rhamnolipid(s), sophorolipid(s), Fatty Acyl Glutamate, Fatty Acyl Glycinate.

The cosmetic cleanser includes the biosurfactants in an amount from approximately 1 to 50 wt. %, preferably approximately 1 to 25 wt. %, more preferably approximately 2 to 15 wt. %, more preferably 3 to 10 wt. %, in relation to the total weight of the cleanser. In the case of biosurfactant mixtures, the specified percentages relate to the total amount of included biosurfactants.

The cosmetic cleanser according to the invention includes, as further necessary constituent, at least one thickening agent of natural origin.

Thickening agents of vegetable origin, such as polysaccharides such as celluloses (cellulose itself and derivatives thereof), alginic acids (and their corresponding physiologically acceptable salts, i.e. alginates), agar agar (with the polysaccharide agarose present as main constituent in agar agar), starch fractions and derivatives such as amylose, amylopectin and dextrins, karaya rubber, gellan gum, locust bean gum, gum arabic, dextrans, guar gum and xanthan gum, or combinations thereof, are included with preference in accordance with the invention as thickening agents of natural origin.

Suitable cellulose derivatives are methyl celluloses, ethyl celluloses, hydroxyalkyl celluloses (such as hydroxyethyl cellulose), methyl hydroxyalkyl celluloses and carboxymethyl celluloses (INCI: Cellulose Gum) and physiologically acceptable salts thereof.

The thickening agent of natural origin is particularly preferably selected from xanthan gum, guar gum, gellan gum, pectin, hydroxymethyl cellulose, or a combination thereof.

In embodiments of the invention, further thickening agents, in particular silicic acids with sufficiently small particle size so as not to act abrasively, can also be included in addition to the thickening agent of natural origin. Further thickening agents that are conventional in exfoliants, such as polyacrylates, can also be included, but are preferably included only in relatively small amounts, and even more preferably are not included at all.

The thickening agent of natural origin is present in an amount from 0.2 to 3 wt. %, preferably 0.3 to 1.5 wt. %, more preferably 0.1 to 10 wt. %, likewise preferably 0.5 to 5 wt. %, in each case in relation to the total weight of the cosmetic cleanser. If a plurality of thickening agents of natural origin are included, the specified percentages relate to the total amount of thickening agents of natural origin.

If silicic acids are also included, these can be included in an amount from approximately 0.5 to 5 wt. %, preferably approximately 1 to 3 wt. %.

In preferred embodiments the cosmetic product according to the invention includes 0.5 wt. % or less of further surfactants, even more preferably no further surfactants, i.e. no further non-ionic, anionic, cationic, amphoteric and/or zwitterionic surfactants. In more preferred embodiments, the cosmetic product according to the invention includes 0.5 wt. % or less, more preferably 0.2 wt. % or less, more preferably no anionic, cationic, amphoteric and/or zwitterionic surfactants. Here, non-ionic surfactants can preferably be included, more preferably in amounts from 0.1 to 10 wt. %, more preferably 0.2 to 5 wt. %, even more preferably 0.5 to 3 wt. %. In these cases, the non-ionic surfactants are preferably non-ionic surfactants based on sugar, such as alkyl polyglycosides, in particular alkyl polyglucosides, and/or fatty acid N-alkyl glucamides, which are also referred to as fatty acyl glucamides. In particular, alkyl polyglycosides based on cured C12/14 coconut alcohol or lauryl alcohol with a DP from 1-3, as are commercially obtainable for example under the INCI names “Coco-Glucoside” or “Lauryl Glucoside”, are preferred alkyl polyglycosides. In particular, preferred fatty acyl glucamides are those that are commercially obtainable under the INCI name Lauroyl Methyl Glucamide, Myristoyl Methyl Glucamide or Lauroyl/Myrstoyl Methyl Glucamide.

The exfoliant according to the invention preferably also includes one or more components as additives, selected from fragrances or perfumes, antioxidants, preservatives, pH regulators, care substances and moisturisers. Other additives, however, can also be included in accordance with the invention.

In particular, potential fillers include natural fillers, possibly water-soluble fillers, such as talc, starch such as maize starch or wheat starch, calcium carbonate, cellulose, and combinations thereof.

Natural fragrances, in particular, are preferred in accordance with the invention as fragrances or perfumes that can be included. If a fragrance is included, this is preferably present in an amount from 0.05 to 3 wt. %, preferably 0.1 to 2 wt. %, more preferably 0.2 to 1 wt. %, and likewise preferably 0.5 to 1 wt. %, in each case in relation to the total weight of the cleanser. If a plurality of fragrances are included, the specified percentages relate to the total amount of fragrances or perfumes.

Typical antioxidants that can be used in accordance with the invention are t-butylhydroxytoluene or vitamin (Tocopherol Acetate).

As care substance, the exfoliant can include, for example, oil bodies, preferably natural oil bodies such as vegetable oils and fats, plant extracts, but also mono- or oligosaccharides and/or lipids. Examples include Aloe vera extracts (INCI: Aloe Barbadensis Leaf Juice), lanolin, niacinamide, avocado oil, or olive oil.

The exfoliant according to the invention can include, for example, glycerol, sorbitol and/or lactates as moisturiser.

### Table Overview:

Preferred cosmetic cleansers will be specified below. The amounts are, in each case, in wt. % and relate to the active substance concentration.

| | Formula 1 | Formula 2 | Formula 3 | Formula 4 |
|---|---|---|---|---|
| Abrasive component | 0.1 to 20 | 1 to 15 | 2 to 15 | 5 to 12 |
| Biosurfactant | 1 to 50 | 1 to 25 | 1 to 20 | 1.5 to 10 |
| Thickening agent | 0.1 to 3 | 0.2 to 2 | 0.2 to 2 | 0.3 to 1.5 |
| Misc | ad 100 | ad 100 | ad 100 | ad 100 |
| | Formula 1a | Formula 2a | Formula 3a | Formula 4a |
| Abrasive component: | 0.1 to 20 | 1 to 15 | 2 to 15 | 5 to 12 |
| Polylactic acid, Sand, | | | | |
| Silicon dioxide, Titanium | | | | |
| dioxide, Jojoba wax, | | | | |
| Cellulose, Comminuted | | | | |
| plant parts and/or | | | | |
| Comminuted fruit stones | | | | |
| Biosurfactant: | 1 to 50 | 1 to 25 | 1 to 20 | 1.5 to 10 |
| Rhamnolipid, | | | | |
| Sophorolipid, Fatty Acyl | | | | |
| Glutamate and/or Fatty | | | | |
| Acyl Glycinate | | | | |
| Thickening agent: | 0.1 to 3 | 0.2 to 2 | 0.2 to 2 | 0.3 to 1.5 |
| Xanthan Gum, Guar Gum and/or | | | | |
| Gellan Gum | | | | |
| Misc | ad 100 | ad 100 | ad 100 | ad 100 |
| | Formula 1b | Formula 2b | Formula 3b | Formula 4b |
| Abrasive component: | 0.1 to 20 | 1 to 15 | 2 to 15 | 5 to 12 |
| Polylactic acid and Sea | | | | |
| sand | | | | |
| Biosurfactant: | 1 to 50 | 1 to 25 | 1 to 20 | 1.5 to 10 |
| Rhamnolipid, | | | | |
| Sophorolipid, Fatty Acyl | | | | |
| Glutamate or Fatty Acyl | | | | |
| Glycinate | | | | |
| Thickening agent: | 0.1 to 3 | 0.2 to 2 | 0.2 to 2 | 0.3 to 1.5 |
| Xanthan Gum, Guar Gum and/or | | | | |
| Gellan Gum | | | | |
| Misc | ad 100 | ad 100 | ad 100 | ad 100 |
| | Formula 5 | Formula 6 | Formula 7 | Formula 8 |
| Abrasive component | 0.1 to 20 | 1 to 15 | 2 to 15 | 5 to 12 |
| Biosurfactant | 1 to 50 | 1 to 25 | 1 to 20 | 1.5 to 10 |
| Thickening agent | 0.1 to 3 | 0.2 to 2 | 0.2 to 2 | 0.3 to 1.5 |
| Non-ionic surfactant | 0.1 to 10 | 0.2 to 5 | 0.3 to 4 | 0.5 to 3 |
| Misc | ad 100 | ad 100 | ad 100 | ad 100 |
| | Formula 5a | Formula 6a | Formula 7a | Formula 8a |
| Abrasive component: | 0.1 to 20 | 1 to 15 | 2 to 15 | 5 to 12 |
| Polylactic acid, Sand, | | | | |
| Silicon dioxide, Titanium | | | | |
| dioxide, Jojoba wax, | | | | |
| Cellulose, Comminuted | | | | |
| plant parts and/or | | | | |
| Comminuted fruit stones | | | | |
| Biosurfactant: | 1 to 50 | 1 to 25 | 1 to 20 | 1.5 to 10 |
| Rhamnolipid, | | | | |
| Sophorolipid, Fatty Acyl | | | | |
| Glutamate and/or Fatty | | | | |
| Acyl Glycinate | | | | |
| Thickening agent: | 0.1 to 3 | 0.2 to 2 | 0.2 to 2 | 0.3 to 1.5 |
| Xanthan Gum, Guar Gum and/or | | | | |
| Gellan Gum | | | | |
| Non-ionic surfactant: | 0.1 to 10 | 0.2 to 5 | 0.3 to 4 | 0.5 to 3 |
| Alkyl polyglycoside | | | | |
| and/or Fatty acyl | | | | |
| glucamide | | | | |
| Misc | ad 100 | ad 100 | ad 100 | ad 100 |
| | Formula 5b | Formula 6b | Formula 7b | Formula 8b |
| Abrasive component: | 0.1 to 20 | 1 to 15 | 2 to 15 | 5 to 12 |
| Polylactic acid and Sea | | | | |
| sand | | | | |
| Biosurfactant: | 1 to 50 | 1 to 25 | 1 to 20 | 1.5 to 10 |
| Rhamnolipid, | | | | |
| Sophorolipid, Fatty Acyl | | | | |
| Glutamate or Fatty Acyl | | | | |
| Glycinate | | | | |
| Thickening agent: | 0.1 to 3 | 0.2 to 2 | 0.2 to 2 | 0.3 to 1.5 |
| Xanthan Gum, Guar Gum and/or | | | | |
| Gellan Gum | | | | |
| Abrasive component: | 0.1 to 10 | 0.2 to 5 | 0.3 to 4 | 0.5 to 3 |
| Polylactic acid and Sea | | | | |
| sand | | | | |
| Non-ionic surfactant: | 0.1 to 10 | 0.2 to 5 | 0.3 to 4 | 0.5 to 3 |
| Lauryl glucoside, | | | | |
| Lauroyl Methyl | | | | |
| Glucamide, Myristoyl | | | | |
| Methyl Glucamide and/or | | | | |
| Lauroyl/Myristoyl | | | | |
| Glucamide | | | | |
| Misc | ad 100 | ad 100 | ad 100 | ad 100 |

“Misc” is to be understood, in accordance with the invention, to mean water, possibly other cosmetic carriers in smaller amounts, and also conventional ingredients of exfoliants, such as fillers, fragrances or perfumes, antioxidants, complexing agents, care substances and/or moisturisers. In embodiments of the invention, “Misc” can also include further thickening agents, in particular silicic acids.

### Examples

The following exfoliants presented in the tables were produced. The specified percentages are to be understood here as percentages by weight, in each case in relation to the total weight of the cleanser.

TABLE 1

| Ingredient | INCI | Example 1 | Example 2 | Example 3 | Example 4 | Example 5 | Example 6 |
|---|---|---|---|---|---|---|---|
| Keltrol SF | Xanthan Gum | 0.80 | — | 0.80 | — | — | — |
| Jaguar S | Guar Gum | — | 0.80 | — | 0.80 | — | — |
| | Gellan Gum | — | — | — | — | 1.00 | 1.00 |
| Sorbitol 70% | Sorbitol, | 15.00 | 15.00 | 15.00 | 15.00 | 15.00 | 15.00 |
| HS DAB | Aqua (Water) | | | | | | |
| Sopholiance S | Sophorolipid | 1.62 | 1.62 | — | — | — | — |
| Rhamnolipid | Rhamnolipid | — | — | 1.04 | 1.04 | — | — |
| R-90 | | | | | | | |
| Lipopeptide | Fatty Acyl | — | — | — | — | 2.50 | — |
| | Glutamate | | | | | | |
| | Fatty Acyl | — | — | — | — | — | 2.50 |
| | Glycinate | | | | | | |
| Plantacare ® | Lauryl | 5.00 | 5.00 | 5.00 | 5.00 | 5.00 | 5.00 |
| 1200 UP | Glucoside | | | | | | |
| Na-benzoate | Sodium | 0.40 | 0.40 | 0.40 | 0.40 | 0.40 | 0.40 |
| | Benzoate | | | | | | |
| Ecoscrub 50 | Polylactic | 3.00 | 3.00 | 3.00 | 3.00 | 3.00 | 3.00 |
| PC | Acid | | | | | | |
| Titanium | CI 77891 | 0.40 | 0.40 | 0.40 | 0.40 | 0.40 | 0.40 |
| Dioxide | (Titanium | | | | | | |
| | Dioxide) | | | | | | |
| Sea Sand | Quartz | 7.00 | 7.00 | 7.00 | 7.00 | 7.00 | 7.00 |
| Citric acid | Citric Acid | 0.20 | 0.30 | 0.50 | 0.25 | 0.74 | 0.74 |
| monohydrate | | | | | | | |
| Syloblanc 34 | Silica | 2.40 | 2.40 | 2.40 | 2.40 | 2.40 | 2.40 |
| Silicic acid, | Silica | 0.50 | 0.50 | 0.50 | 0.50 | 0.50 | 0.50 |
| hydrophilic | | | | | | | |
| BET | | | | | | | |
| Water, | | ad 100 | ad 100 | ad 100 | ad 100 | ad 100 | ad 100 |
| demineralised | | | | | | | |

TABLE 2

| | | | | | Example |
|---|---|---|---|---|---|
| Ingredient | INCI | Example 7 | Example 8 | Example 9 | 10 |
| Keltrol SF | Xanthan Gum | — | — | 0.80 | 0.80 |
| Jaguar S | Guar Gum | 0.80 | 0.80 | — | — |
| Sorbitol 70% HS | Sorbitol, Aqua | 15.00 | 15.00 | 15.00 | 15.00 |
| DAB | (Water) | | | | |
| Lipopeptide | Fatty Acyl | — | 0.945 | 0.945 | 0.945 |
| | Glutamate | | | | |
| Lipopeptide | Sodium Lauroyl | 5.00 | — | — | — |
| | Glutamate | | | | |
| Glucotain Flex | Lauroyl/Myristoyl | — | — | — | 5.00 |
| | Methyl Glucamide | | | | |
| Plantacare ® | Lauryl Glucoside | — | 5.00 | 5.00 | — |
| 1200 UP | | | | | |
| Na-benzoate | Sodium Benzoate | 0.40 | 0.40 | 0.40 | 0.40 |
| Ecoscrub 50 PC | Polylactic Acid | 3.00 | 3.00 | 3.00 | 3.00 |
| Titanium | CI 77891 | 0.40 | 0.40 | 0.40 | 0.40 |
| Dioxide | (Titanium Dioxide) | | | | |
| Sea Sand | Quartz | — | 7.00 | 7.00 | 7.00 |
| Jojoba wax | Hydrogenated | 7.00 | — | — | — |
| beads | Jojoba Oil | | | | |
| Citric acid | Citric Acid | 0.80 | 0.80 | 0.76 | 0.76 |
| monohydrate | | | | | |
| Syloblanc 34 | Silica | 2.40 | 2.40 | 2.40 | 2.40 |
| Silicic acid | Silica | 0.50 | 0.50 | 0.50 | 0.50 |
| hydrophilic BET | | | | | |
| Water, | | ad 100 | ad 100 | ad 100 | ad 100 |
| demineralised | | | | | |

All exfoliants have excellent cleansing and foaming behaviour with a pleasant feel to the skin, although natural or at least easily biologically degradable components were used largely or exclusively.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleanser including the following constituents, in each case in wt. % in relation to the total weight:
(a) 0.1 to 20 wt. % of at least one water-insoluble abrasive component of natural origin,
(b) 1 to 50 wt. % of at least one biosurfactant, and
(c) 0.2 to 3 wt. % of at least one thickening agent of natural origin.

2. The cosmetic cleanser according to claim 1, including
(a) 1 to 15 wt. % of the abrasive component,
(b) 2 to 20 wt. % of the biosurfactant, and
(c) 0.3 to 1.5 wt. % of the thickening agent.

3. The cosmetic cleanser according to claim 1, the water-insoluble abrasive component being one or more selected from the group consisting of particles of polylactic acid, sand, silicon dioxide, titanium dioxide, jojoba wax, cellulose, comminuted plant parts, and comminuted algae.

4. The cosmetic cleanser according to claim 1, which includes a glycolipid, a lipopeptide, or a combination thereof as biosurfactant.

5. The cosmetic cleanser according to claim 1, which includes a rhamnolipid, a sophorolipid, a mannosylerythritol, a surfactin, a Fatty Acyl Glutamate, Fatty Acyl Glycinate, or a combination thereof as the biosurfactant.

6. The cosmetic cleanser according to claim 5, which includes a sophorolipid as the biosurfactant, 20 to 60 wt. % being present as acid form.

7. The cosmetic cleanser according to claim 5, which includes a mixture of mono- and dirhamnolipid as the biosurfactant, which are each derived from 3-hydroxydodecanoic acid and 3-hydroxyundecanoic acid.

8. The cosmetic cleanser according to claim 1, the thickening agent (c) being selected from the group consisting of xanthan gum, guar gum, gellan gum, pectin, hydroxymethyl cellulose, and a combination thereof.

9. The cosmetic cleanser according to claim 1, which also includes a non-ionic surfactant of natural origin selected from the group consisting of alkyl polyglycoside(s), fatty acyl glucamide(s), and a combination thereof.

10. The cosmetic cleanser according to claim 1, which includes less than 0.5 wt. % anionic, amphoteric/zwitterionic and/or cationic surfactant.

11. The cosmetic product according to claim 1, which further includes one or more components selected from the group consisting of fragrances or perfumes, antioxidants, complexing agents, care substances and moisturisers.

12. The cosmetic product according to claim 1, which is formulated as a facial cleanser or as a body cleanser.
